(19) United States
(12) Patent Application Publication
(10) Pub. No.: US 2013/0228169 A1
(43) Pub. Date: Sep. 5, 2013

(54) AEROSOL THERAPY DEVICE

(71) Applicant: PARI PHARMA GMBH, Starnberg (DE)

(72) Inventor: Roland STANGL, Moosburg (DE)

(73) Assignee: PARI PHARMA GMBH, Starnberg (DE)

(21) Appl. No.: 13/848,502

(22) Filed: Mar. 21, 2013

Related U.S. Application Data
(63) Continuation of application No. 10/527,835, filed on Sep. 6, 2005, now abandoned.

(30) Foreign Application Priority Data
Sep. 18, 2002 (DE) 102 43 371.2

(51) Int. Cl. A61M 11/04

(52) U.S. Cl. CPC A61M 11/04
USPC 128/200.23

(57) ABSTRACT

The invention relates to an aerosol therapy device comprising a membrane nebulizer (1) and a personal digital assistant (PDA (2)), which are interconnected via communication devices (10, 11) in such a way that therapy data, which has been prepared in relation to signals, is transmitted from the membrane nebulizer (1) to the PDA and control data is transmitted from the latter (2) to the membrane nebulizer (1).

This application is a Continuation of U.S. application Ser. No. 10/527,835 filed 6 Sep. 2005, which is a National Stage Application of PCT/EP2003/010405, filed 18 Sep. 2003, which claims benefit of Serial No. 102 43 371.2, filed 18 Sep. 2002 in Germany and which applications are incorporated herein by reference. A claim of priority to all, to the extent appropriate is made.

### DESCRIPTION

The invention relates to an aerosol therapy device comprising a nebuliser and a PDA (Personal Digital Assistant).

Personal digital assistants, hereinafter PDA, have been known for quite some time and have an ever-increasing efficiency. Within the context of the following description, PDA is to be understood as a small computer, the dimensions of which correspond approximately to those of a pocketbook. A PDA generally comprises a processor, a memory, which is configured partly as a non-volatile memory, a display means, which is generally configured as a graphic display, and an input means, which can be configured, for example, as a keypad or as a touch-sensitive surface in the graphic display. A PDA often comprises further interfaces, for example slots for memory cards and/or ports for remote data connections. A PDA comprises an operating system as well as different, generally useable application programs which are stored in the non-volatile part of the memory. In the case of a PDA of the type in question here, it is essential that the operating system allows further programs to be executed, which can be loaded for this purpose into the memory of the PDA or are provided via a memory card in a slot of the PDA. These further programs are carried out within the framework of the operating system of the PDA or replace it completely. PDA's of the type in question here are generally operated independently of a network, but can, however, also additionally comprise a port for a network-linked power supply.

Various examples for the use of PDA's in connection with medical devices for personal use are known from the prior art. Cited as examples at this point are EP 0 617 628 B1, WO 01/214690 A2, WO 01/28416 A1 and WO 01/5272. These examples are characteristic for the prior art in as much as the use of the PDA in medical-technical devices is generally restricted to the receiving and transfer of data from the medical-technical device as well as the evaluation and display of the received data on the display means of the PDA.

This restriction of the use of PDA's in medical-technical devices is explained in the following by means of the aforementioned publication WO 01/24690 A2. In this publication, a spray-burst nebuliser is described, from which a medicament, which is stored in a container, can be removed in the form of a spray-burst aerosol. The spray-burst nebuliser is hand-operated, however it comprises an electronic data processing unit which displays information to the patient about the therapy being carried out with the spray-burst nebuliser. For this purpose, the electronic data processing unit collects information about the manually performed applications of the spray-burst aerosol. The data processing unit of the spray-burst nebuliser is able to establish a connection with a PDA via an interface in order to transmit and receive data. The PDA serves as an interface to a computer network, via which the data reaches specialised servers and/or the treating doctor. Following evaluation of the transmitted data, the therapy being carried out with the spray-burst nebuliser can be influenced. Information is transmitted for this purpose by the PDA to the electronic data management system of the spray-burst nebuliser and is displayed to the patient. The patient adapts the therapy in accordance with the information displayed.

As in the case of the last described example of the prior art, the use of a PDA in a medical therapy device is always restricted to the evaluation of data, on the basis of which information is ascertained which is displayed to the patient. It is the patient's responsibility to convert this information into a corresponding modification of the therapy, for example by amending the dose. It must be stated with regard to the cases of use known to date that it cannot be ensured due to the system that the patient will correctly convert the transmitted information and instructions and will correspondingly adapt the therapy.

Against this background, the invention wants to provide a way to eliminate the current system-immanent gaps and to realise a reliable therapy modification when using a PDA, particularly when interacting with an aerosol therapy nebuliser.

The present invention extends beyond the known prior art and allocates the PDA an evaluation and control function in addition to the known functions. The system according to the invention thus differs from the prior art in that the PDA not only evaluates the received data in relation to the therapy, but rather also acts on the therapy nebuliser in a controlling manner. According to the invention, the nebuliser must be configured for this purpose such that it is possible for the PDA to intervene in a controlling manner. This means, on the one hand, that the nebuliser is configured to receive control information from the PDA and that the aerosol generator is controllable. On the other hand, this also means that the nebuliser is basically able to process control information when generating the aerosol and to influence generation of the aerosol in accordance with the control information. Electrically operated aerosol generators are particularly suitable for the controlled generation of an aerosol. As regards electrically operated aerosol generators, membrane aerosol generators must in turn be emphasised owing to their very good controllability, in which a membrane is caused to oscillate by means of an electrically excited piezo element. The aforementioned spray-burst nebuliser according to WO 01/24690 A2 and many other types of nebuliser are, on the other hand, not suitable or are only suitable to an unsatisfactory extent for a use according to the invention owing to a lack of controllability.

According to the invention, this possibility is created in that the nebuliser is configured with a controllable aerosol generator and preferably as a membrane nebuliser, which is equipped with a suitable interface for communication in order to transmit data to the PDA and to receive control information. This control information can be immediately taken into consideration in a controllable nebuliser, in particular a membrane nebuliser, during the therapy session and can be incorporated during operation such that in the case of the pairing according to the invention, i.e. of a controllable nebuliser, in particular a membrane nebuliser, with a PDA, far more functions, namely evaluation and control functions based on the evaluated data, can be allocated to the PDA. Thus, very complicated evaluations can be carried out and the results thereof can be used in a controlling manner without it being necessary to equip the membrane nebuliser with a correspondingly complex arithmetic unit. The controllable nebuliser, in particular the membrane nebuliser according to the invention, can be constructed comparatively simply provided that the transfer of data to the PDA and the receipt of control information from the PDA as well as the implementation thereof for the control of aerosol generation are ensured. In order to ensure a basic operation of the membrane nebuliser, construction should, however, occur such that the membrane nebuliser can also be used without the PDA, even if the control processes only realised by means of the PDA thereby have to be abandoned.

The relationship according to the invention between the nebuliser on the one hand and the PDA on the other can be characterised best by the functions allocated to the two components each as a special feature according to the invention. As regards the controllable nebuliser, it is decisive according to the invention that a signal-based processing of measuring signals occurs such that data relevant for the therapy can be transmitted to the PDA. The PDA in turn conducts a therapy-related evaluation of the data, at the end of which control information is acquired which is transmitted to the controllable nebuliser so as to have a direct effect on the therapy session. Thus, the system according to the invention consisting of a controllable nebuliser and a PDA can be characterised on the one hand by a signal-based processing of measuring signals to transfer therapy data and on the other by a therapy-related evaluation to prepare/transfer control information influencing the therapy. This pairing of components and allocation of functions is not known in the prior art, even though many systems were described in which a PDA was combined with a medical-technical device or even with a nebuliser.

Against this background, “signal-based processing” is to be understood in the following as all measures and means provided in the membrane nebuliser in order to obtain and prepare sensor signals, i.e., for example to filter them, smooth them, amplify them, digitalise them, etc. The sensor signals are mostly processed using electrotechnical means regardless of the therapeutic background. In contrast hereto, “therapy-related evaluation” is to be understood as all those measures and means that are realised in the PDA to examine the data in respect of the therapy so that conclusions regarding the therapy can be made on the basis of the examination results and the therapy can thus be influenced in a controlling manner based on these results.

The invention will be described in more detail below by means of an embodiment and referring to the figures, in which

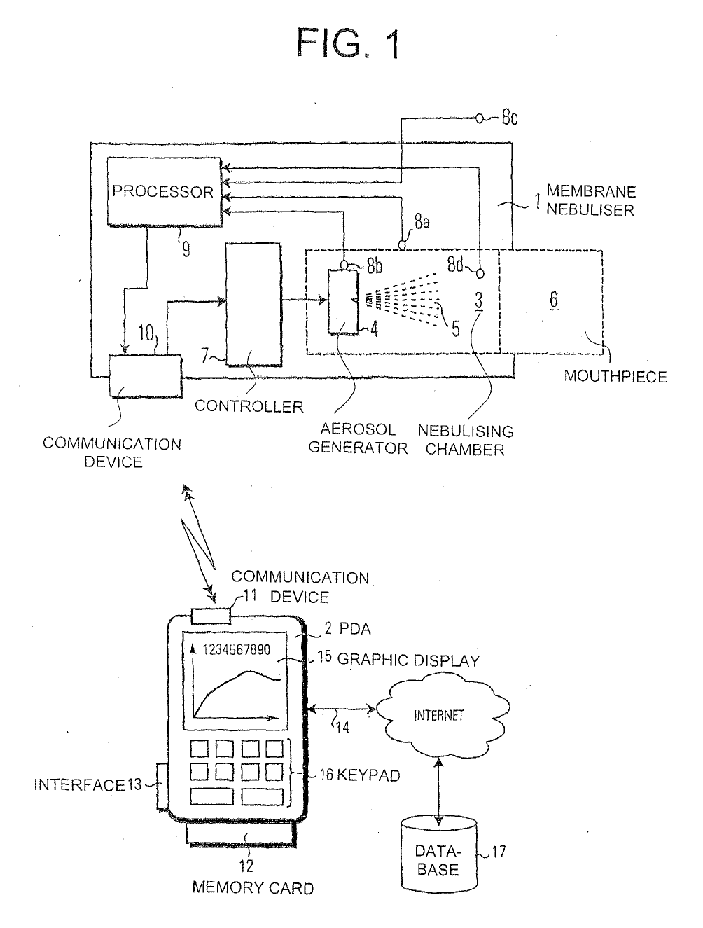

FIG. 1 shows a schematic diagram of an aerosol therapy device according to the invention having a membrane nebuliser and a PDA;

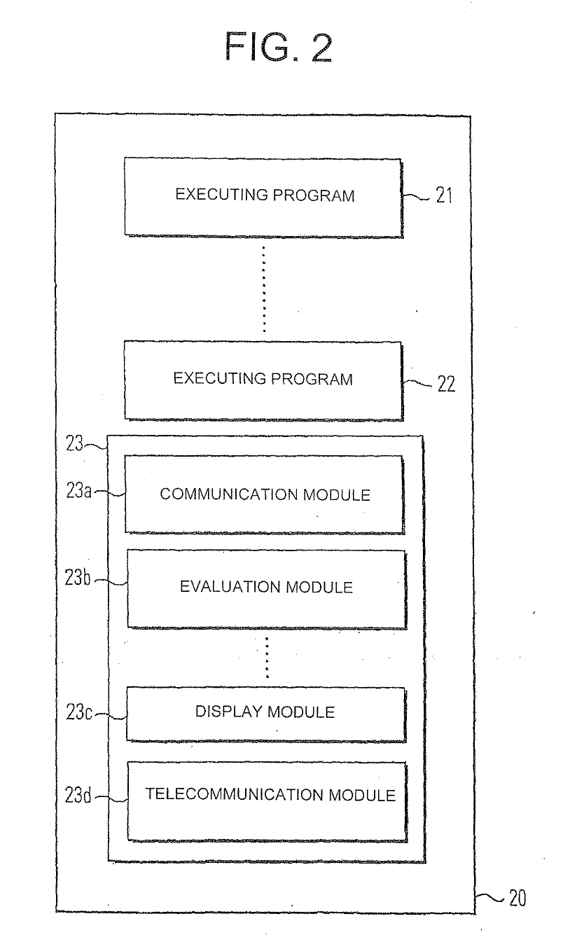

FIG. 2 shows a schematic diagram of the program module according to the invention in the PDA;

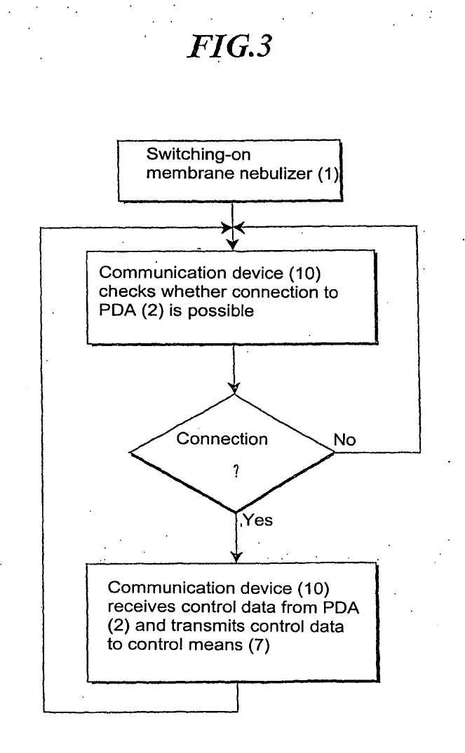

FIG. 3 shows a flow diagram to demonstrate the processes in the membrane nebuliser according to the invention;

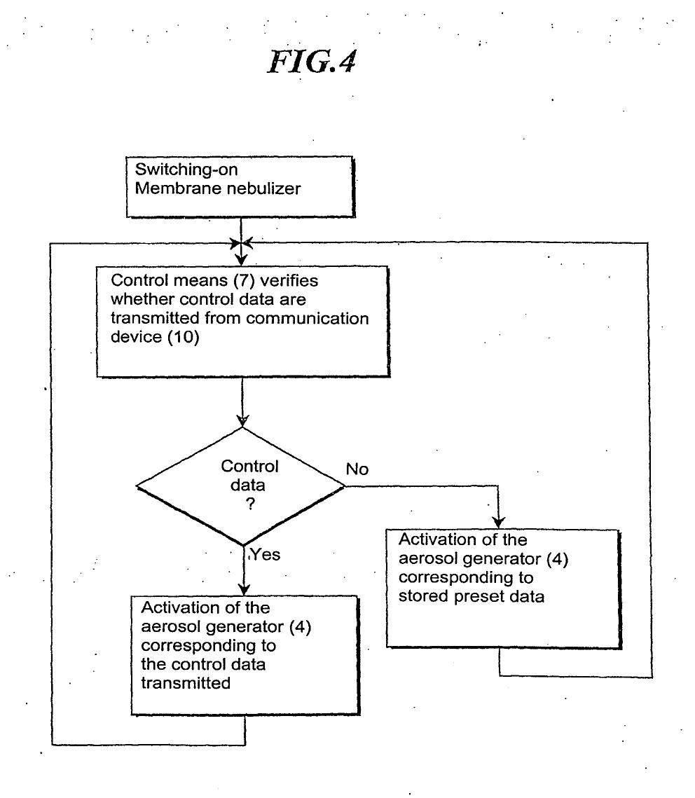

FIG. 4 shows a flow diagram of a further process in the membrane nebuliser according to the invention; and

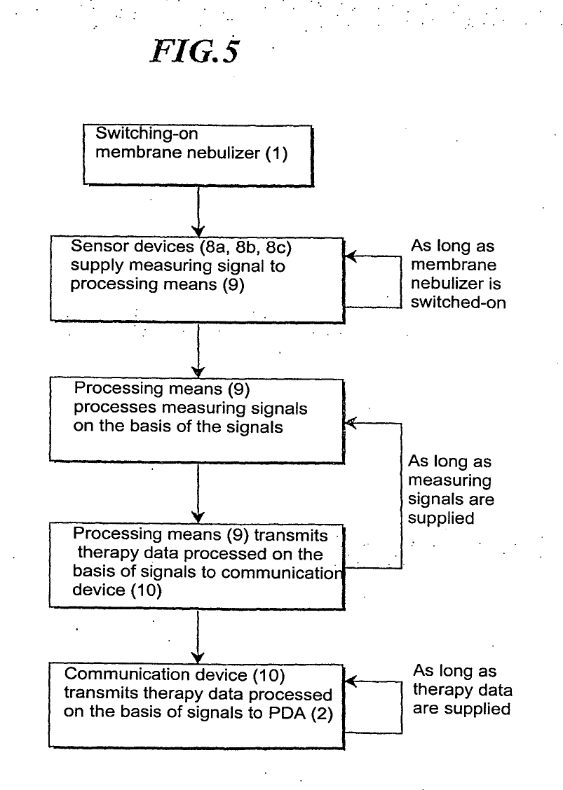

FIG. 5 shows a flow diagram to demonstrate a further operating process in the membrane nebuliser according to the invention.

FIG. 1 schematically shows an aerosol therapy device according to the invention having a membrane nebuliser 1 and a PDA 2. The membrane nebuliser 1 comprises a nebulising chamber 3, into which a membrane aerosol generator 4 generates an aerosol 5. The construction of the membrane aerosol generator 4 corresponds, for example, to the membrane aerosol generator known from DE 199 53 317 C1. The details of the membrane aerosol generator 4 and of the corresponding liquid reservoir are not shown in the schematic diagram of the embodiment in FIG. 1. A patient can inhale the aerosol 5 generated in the nebulising chamber 3 via a mouthpiece 6. The nebulising chamber is preferably designed such that during inhalation, ambient air can flow in. In order to generate the aerosol 5, the membrane aerosol generator 4 is activated by a control means 7.

In the embodiment shown in FIG. 1, the membrane nebuliser 1 comprises three sensor devices 8a, 8b and 8c. The sensor devices 8a, 8b and 8c supply measuring signals of parameters relevant for the therapy to a processing means which generates therapy data from said measuring signals by means of a signal-based processing of the measuring signals. The therapy data is released to a communication device 10 by the processing means 9 so as to be transmitted via the communication device 10 to the PDA 2.

The sensor devices 8a, 8b, 8c and 8d shown in FIG. 1 represent examples of different sensor devices which generate measuring signals which are relevant for the therapy. For example, sensor device 8a detects the presense of aerosol and the aerosol density in the nebulising chamber 3. The sensor device 8b detects the state of the membrane aerosol generator 4, for example the temperature and current consumption thereof, the filling status of the liquid reservoir or another therapy-relevant parameter of the aerosol generator 4. The sensor device 8c detects, for example, the temperature, humidity or the pressure of the ambient air. Sensor device 8d detects, for example, the respiratory flow of the patient. Although four sensor devices 8a, 8b, 8c and 8d are shown in the embodiment according to FIG. 1, it is obvious that an aerosol therapy device according to the invention can be equipped with more or less sensor devices for generating more or less therapy-relevant measuring signals. It is important that at least one therapy-relevant measuring signal from at least one sensor device is supplied to the processing means 9 to generate therapy data by means of the signal-based processing of sensor signals.

The therapy data obtained by means of signal-based processing is conveyed to the communication device 10 by the processing means 9 for transfer to the PDA 2. The communication device 10 transfers the therapy data which has been processed in relation to signals to the PDA 2 which comprises a compatible communication device 11 for this purpose. The communication devices 10 and 11 are typically IR (infrared) communication devices or radio communication devices, for example according to the Blue-Tooth standard, or another suitable communication link between the membrane nebuliser 1 and the PDA 2, for example a cable connection. It is only important that the communication devices 10 and 11 enable bi-directional communication between the membrane nebuliser 1 and the PDA 2. This is because in addition to the transfer of the therapy data processed in relation to signals from the membrane nebuliser 1 to the PDA 2, control data is also transferred according to the invention from the PDA 2 to the membrane nebuliser 1. This control data is transferred from the communication device 11 of the PDA 2 to the communication device 10 of the membrane nebuliser 1 and is conveyed to the control means 7 by the communication device 10 of said membrane nebuliser 1. The control means 7 activates the membrane aerosol generator 4 in accordance with the supplied control data.

A device for generating the control data by means of the therapy-related evaluation of the transmitted therapy data is realised in the PDA 2, preferably in the form of a program, which accepts the therapy data that has been processed in relation to signals and is received by the communication device 11 of the PDA 2, and which evaluates said data in relation to the therapy. The invention thereby uses the property of the PDA 2 that it can execute any programs.

The therapy-related evaluation at least provides control data according to the invention, which is transmitted to the membrane nebuliser 1 via the communication device 11 of the PDA 2. The programmability of the PDA 2 makes it possible to realise almost any therapy-related evaluations which can be selected depending on the medicament used, the therapy desired and other limiting conditions.

The PDA 2 of the aerosol therapy device according to the invention can be equipped with a program for therapy-related evaluation in different manners. As shown in FIG. 1, the program can be stored on a memory card 12 (for example Compact Flash, Smart Media, Memory Stick, or similar cards), which is inserted into a corresponding receiving slot of the PDA 2, such that a program stored on the memory card 12 can be executed within the scope of the operating system of the PDA 2. A program can also be transferred from a memory card 12 into the main memory of the PDA 2 and can be started from there within the framework of the operating system of the PDA 2. Furthermore, it is alternatively possible to transfer a program into the main memory of the PDA 2 via an interface, for example the communication device 11 or an additional interface 13 (RS-232, USB, FireWire, or the like) and to start it from there within the framework of the operating system of the PDA 2. Finally, as shown in FIG. 1, a program can be loaded into the main memory of the PDA 2 via a remote data connection means 14, for example via the Internet, and can be started from there within the framework of the operating system of the PDA 2.

The operating system 20 of the PDA 2 is schematically shown in FIG. 2. The operating system 20 offers the possibility of executing programs 21, 22 in any manner within the framework of the operating system 20 of the PDA 2. The operating system 20 thereby assumes control, in particular of the input/output interfaces, for example of a graphic display 15 and a keypad 16, and also of the communication device 11 of the PDA 2. The programs 21, 22 executed within the framework of the operating system 20 can access the interfaces via the operating system 20. The program 23 according to the invention, as shown in FIG. 2, is also executed within the framework of the operating system 20 of the PDA and can thereby access, via the operating system 20, the communication device 11 for receiving the therapy data processed in relation to signals and for transmitting the control data. The program 23 according to the invention preferably comprises a communication module 23a for this purpose, which undertakes these functions. The therapy data received by the communication module 23a via the communication device 11 is evaluated in an evaluation module 23b and control data is generated which is conveyed to the communication module 23a and which is transmitted to the membrane nebuliser 1 via the communication device 11 with the help of the operating system 20.

The program 23 according to the invention can contain further modules, for example a module 23c for displaying therapy data and/or control data on the graphical display 15 of the PDA 2. The therapy data can be displayed to the user in the form of alphanumerical characters or in the form of diagrams or in another manner by means of the display 15 of the PDA.

A telecommunication module 23d can undertake the function of creating a connection to a databank 17 via the remote data connection means 14, for example via the Internet, in order to transfer therapy data and/or control data. Furthermore, the program module 23d can retrieve other program modules from a databank 17 via the remote data connection, for example via the Internet, and store them in the main memory or on a memory card 12 of the PDA 2 for use within the framework of the program 23 according to the invention and the operating system 20 of the PDA 2.

The construction of the program 23 according to the invention in the form of program modules 23a to 23d as described above is only an example. The program 23 according to the invention can also be realised as an individual program module which combines all the functions therein and is carried out within the framework of the operating system 20 of the PDA 2.

It is possible for the user to enter data via a keypad 16 of the PDA 2, which is taken into consideration during the therapy-related evaluation of the transmitted therapy data processed in relation to signals or when displaying the therapy data processed for display on the display 15 of the PDA.

It will be described as an example below, which operating processes there are in an aerosol therapy device according to the invention.

As shown in FIG. 3, following switching on of the membrane nebuliser 1, the communication device 10 of the membrane nebuliser verifies whether or not a communication link can be established to the PDA 2 in order to receive control data from said PDA 2. As long as a communication link to the PDA 2 is not established, the communication device 10 of the membrane nebuliser 1 will continue verification. The communication device 10 resumes verification if an established communication link to the PDA 2 is interrupted. During verification of the communication link, the communication device 10 does not transmit any control data to the control means 7. If a communication link with the PDA 2 exists and control data is being transmitted, the communication device 10 transmits this control data to the control means 7 of the membrane nebuliser 1.

As shown in FIG. 4, following switching on of the membrane nebuliser 1, the control means 7 verifies whether or not control data is being supplied by the communication device 10 of the membrane nebuliser 1. If no control data is being supplied, the control means 7 will control the aerosol generator 4 in accordance with preset control data which is preferably stored in the control means 7. For example, the preset control data can ensure that the control means 7 continuously activates the membrane generator 4 such that an aerosol 5 is continuously generated in the nebulising chamber 3, which can be inhaled by a patient via the mouthpiece 6. If control data is being supplied to the control means 7 by the communication means 10, the control means will control the membrane generator 4 in accordance with this control data transmitted by the PDA 2.

As shown in FIG. 5, following switching on of the membrane nebuliser 1, the sensor devices 8a, 8b and 8c emit sensor signals to the processing means 9, which generates therapy data from the measuring signals by means of a signal-based processing of said measuring signals and conveys this data to the communication device 10. If the communication device 10 receives therapy data that has been processed in relation to signals from the processing means 9, the communication device 10 will transmit this data to the PDA 2.

The sensor devices, the processing means and the communication devices work independently to the extent that they carry out the function allocated to them as long as the initial conditions are met. That is to say the sensor devices 8a, 8b, 8c will supply sensor signals provided that the membrane nebuliser 1 is switched on. The evaluation means 9 will process the received measuring signals and convey them to the communication device 10 as long as measuring signals are being supplied. The communication device 10 will transmit the processed data to the PDA 2 as long as data processed in relation to signals is being supplied by the processing means 9.

The PDA 2 generates the control data by means of the therapy-related evaluation of the therapy data processed in relation to signals which is transmitted by the communication device 10 of the membrane nebuliser 1. If no therapy data processed in relation to signals and transmitted via the communication devices 10 and 11 of the membrane nebuliser and the PDA is present in the PDA 2, the PDA 2 will transmit preset control data corresponding, for example, to the preset control data stored in the control means 7. However, other preset control data can also be stored in the PDA, which is transmitted to the membrane nebuliser 1 and is supplied to the control means 7 of the membrane nebuliser 1 by the communication means 10. As long as the membrane nebuliser 1 is transmitting, via the communication means 10, therapy data processed in relation to signals to the PDA 2 via its communication means 11, the control data will be generated in the PDA 2 by means of therapy-related evaluation of the transmitted therapy data and will be transmitted to the membrane nebuliser. In this manner, the PDA 2 becomes an integral component of the respiratory therapy device according to the invention since the therapy data processed in relation to signals is evaluated in the PDA in respect of the therapy and control data is generated which is transmitted to the membrane nebuliser for activating the aerosol generator 4.

The control data transmitted by the PDA 2 to the membrane nebuliser 1 can be of different types. It could be, for example, data which determines the duration of aerosol production by the aerosol generator 4. The transferred control data could include data which determines the oscillation frequency of the membrane aerosol generator 4. In this manner, the operating frequency can be adapted to changing limiting conditions which are detected, for example, by the sensors 8a, 8b and 8c. The control data could include data which determined the length of the therapy session if the therapy has to be extended or shortened subject to the limiting conditions. A further sensor could be used in this regard, by means of which the respiration of the patient is detected, which enables, for example, assessment of the deposition behaviour. The control data could furthermore include data by means of which the control means is transferred, at least intermittently, into a service mode so as to check the operating state and the operating ability of the membrane aerosol generator 4. As already stated above, the control data listed above are merely examples and the use of the concept according to the invention is not limited to these types of control data.

1. An aerosol therapy system having a nebulizer, a PDA and a databank, wherein:
a) said nebulizer comprises:
i) a controllable aerosol generator; and
ii) at least one sensor for generating at least one therapy-relevant measuring signal during therapy related to aerosol production and aerosol consumption; and
iii) a processor for generating therapy data by the signal-based processing of at least one measuring signal;
iv) a communication device for transferring the therapy data processed in relation to signals from the nebulizer to the PDA and for receiving control data transmitted from the PDA to the nebulizer; and
b) said databank being external to said nebulizer and said PDA;
c) said PDA comprises:
i) a communication device for receiving the therapy data transmitted by the nebulizer and for transferring control data to the nebulizer; and
ii) a control device for receiving control data from said databank and/or generating control data by a therapy-related evaluation of the transmitted therapy data; and
iii) a telecommunication module for establishing a remote data connection for transmitting therapy and/or control data and/or receiving control data and/or program modules for the PDA and/or the nebulizer;
wherein the communication devices enable a communication link between the nebulizer and the PDA based on radio emissions,
and wherein said PDA is configured to load an external program via said remote data connection into a main memory of the PDA, said program being configured to access the communication device of the PDA for receiving said therapy data and transmitting said control data.

2. An aerosol therapy system according to claim 1, wherein said controllable aerosol generator is a controllable membrane aerosol generator.

3. An aerosol therapy system according to claim 1, wherein said nebulizer comprises a controller for verifying in whether or not control data is received from the PDA, and if control data is received for activating and controlling said aerosol generator, wherein the controller controls the aerosol generator based on the control data, and wherein, if no control data is received from the PDA, the controller controls the aerosol generator based on preset or stored control data such that the nebulizer can be used without the PDA.

4. An aerosol therapy system according to claim 1, wherein said processor of said nebulizer is adapted to generate therapy data further based on at least one detected state of said membrane aerosol generator.

5. An aerosol therapy system according to claim 1, wherein said control device of said PDA is further adapted to generate control data suitable to modify the therapy of the membrane aerosol generator.

6. An aerosol therapy system according to claim 1, wherein the nebulizer further comprises a nebulizing chamber.

7. An aerosol therapy system according to claim 4, wherein said nebulizer further comprises a sensor in said nebulizing chamber.

8. An aerosol therapy system according to claim 1, wherein the controllable membrane aerosol generator is an electrically operated membrane aerosol generator, wherein the membrane is caused to oscillate by means of an electrically excited piezo element.

9. An aerosol therapy system according to claim 1, wherein the sensor comprises a sensor device for detecting the presence and/or the density of an aerosol generated by the aerosol generator and/or a sensor device for detecting the operating state of the aerosol generator and/or a sensor device for detecting the environmental conditions and/or a sensor device for detecting the respiratory flow of the user.

10. An aerosol therapy system according to claim 1, wherein the device for generating control data by the therapy related evaluation of transmitted therapy data comprises a program which can be executed within a framework of an operating system of the PDA.

11. An aerosol therapy system according to claim 10, wherein the comprises program modules, of which at least one module carries out the evaluation of the therapy data processed in relation to signals and generation of control data.

12. An aerosol therapy system according to claim 10, wherein a program module carries out the evaluation of the therapy data processed in relation to signals and generation of control data.

13. An aerosol therapy system according to claim 10, wherein a communication module is provided for receiving therapy data processed in relation to signals and for transmitting control data via the communication device of the PDA.

14. An aerosol therapy system according to claim 10, wherein a further program module and/or output interface is provided for displaying therapy and/or control display data and/or bi-directional communication on a display of the PDA.

15. An aerosol therapy system according to claim 10, wherein a telecommunication module is provided for establishment of a remote data connection for transmitting therapy and/or control data and/or for receiving program modules.

16. An aerosol therapy system according to claim 10, wherein the program and/or one of the program modules is stored on a memory card which can be inserted into a slot of the PDA.

17. An aerosol therapy system according to claim 1, wherein the controller activates the aerosol generator based on control data stored in said controller if the controller is not receiving any control data from the communication device.

18. An aerosol therapy system according to claim 15, wherein the remote data connection comprises the Internet and/or a connection to a databank.

19. An aerosol therapy system according to claim 1, wherein the control data further include data by which the controller is transferred, at least intermittently, into a service mode to check the operating state and/or operating ability of the aerosol generator.

20. An aerosol therapy system according to claim 1, wherein the input interface of the PDA is a keypad and/or touch-sensitive surface for entering data taken into consideration during therapy-related evaluation of the transmitted therapy data.

21. An aerosol therapy system according to claim 1, wherein the control data include data which determine the oscillation frequency of the aerosol generator.

22. An aerosol therapy system according to claim 1, wherein the control data include data which determine the length of the therapy session if the therapy has to be extended or shortened subject to limiting conditions detected by the at least one sensor.

23. An aerosol therapy system according to claim 2, wherein the sensor comprises a sensor device for detecting presence and density of an aerosol generated by the membrane aerosol generator.

24. An aerosol therapy system according to claim 2, wherein the nebulizer having a liquid reservoir which corresponds with the membrane aerosol generator.

25. An aerosol therapy system according to claim 1, wherein the sensor comprises a sensor device for detecting filling status of the liquid reservoir of the nebulizer.

26. An aerosol therapy system according to claim 1, wherein the telecommunication module for establishing a remote data connection transmits therapy and/or control data to said databank.

27. An aerosol therapy system according to claim 1, wherein the telecommunication module establishes a remote data connection receiving control data and/or program modules from said databank or the internet.
